(19) United States
(12) Patent Application Publication
(10) Pub. No.: US 2010/0178335 A1
(43) Pub. Date: Jul. 15, 2010

(54) Formulations of acetylsalicylic acid or its derivatives in soft capsules, exhibiting high stability

(76) Inventors: Angel Mateo Echanagorria, Segrate (IT); Maurizio Marchiorri, Valbrona (IT); Giorgio Zoppetti, Milano (IT)

Correspondence Address: STETINA BRUNDA GARRED & BRUCKER, 75 ENTERPRISE, SUITE 250, ALISO VIEJO, CA, 92656, US

(21) Appl. No.: 12/310,634

(22) PCT Filed: Aug. 30, 2007
(86) PCT No.: PCT/EP2007/059047

§ 371 Date: Feb. 27, 2009

(30) Foreign Application Priority Data
Sep. 1, 2006 (IT) MI2006A001672

(51) Int. Cl. A61K 31/60; A61K 9/66; A61K 9/48; A61P 29/00

(52) U.S. Cl. 424/452; 424/455; 514/165

(57) ABSTRACT

The present invention relates to new formulations comprising acetylsalicylic acid or its derivatives an oil phase and a cyclodextria in soft capsules, characterized by a high stability.

### FIELD OF THE INVENTION

The present invention concerns the field of new pharmaceutical formulation provision and relates to new formulations provided with high patient compliance and exhibiting an improved stability.

### PRIOR ART

Acetylsalicylic acid (or aspirin) is a long known active principle, traditionally used as an anti-inflammatory/analgesic and also more recently as a platelet anti-aggregation agent (so-called aspirin cardio).

With regard to pharmaceutical formulations comprising acetylsalicylic acid, during more than a century of its use a vast number have been developed and on a massive scale. However, the known tendency of acetylsalicylic acid to hydrolyze easily^{[1,2,3]} gives rise to the problem of quasi-spontaneous deterioration of the active principle, which is difficult to control.

Accordingly, the need exists for developing formulations of greater stability whose acetylsalicylic acid content is more constant over time.

Other compounds having long been used with success in the prevention and/or therapy of cardiovascular diseases are the so-called omega-3 oils and their derivatives, which are long chain polyunsaturated carboxylic acids contained in fish oils.

Application no. PCT/EP2006/060649, claiming the priority of the application No. MI2005A000387 filed on the 11 Mar. 2005 by the same applicant, describes among other things aspirin formulations in soft capsules of higher bioavailability, whose gelatin shell comprises cyclodextrins. The components of the oil phase contained in the capsule also include omega-3 oils.

European application EP 1352648 by the same applicant describes compositions in soft capsules in which aspirin and omega-3 acids, or derivatives thereof, are advantageously combined from the therapeutic viewpoint. Compared to known formulations, using soft capsules is also advantageous from the patient compliance viewpoint because of ease of swallowing. It appears from the same application that using omega-3 acids or their derivatives as components of the internal liquid or semi-liquid phase can increase the stability of acetylsalicylic acid thus formulated.

However, the stability of formulations of acetylsalicylic acid or its pharmaceutically acceptable derivatives in soft capsules is not yet satisfactory. Therefore, the technical problem faced and resolved by the present invention is that of providing new formulations of acetylsalicylic acid and its derivatives in soft capsules, in which the acetylsalicylic acid or its pharmaceutically acceptable derivatives is comprised within an internal liquid or semi-liquid oil phase contained therein and in which the acetylsalicylic acid or its pharmaceutically acceptable derivatives are more protected against hydrolysis.

### SUMMARY OF THE INVENTION

The inventors of the present invention have surprisingly found that compounds of the cyclodextrin class are able to stabilize formulations of acetylsalicylic acid or its pharmaceutically acceptable derivatives in soft capsules (or SECS, soft elastic capsules) against hydrolysis of the acetylsalicylic acid or its derivatives “from a distance”, i.e. under conditions that do not promote (or that minimize) the formation of inclusion complexes of cyclodextrins with acetylsalicylic acid or its derivatives within the pharmaceutical formulation in question, i.e. the soft capsule. Conditions that do not promote (or that minimize) complexing of acetylsalicylic acid within the capsule include firstly (a) the spatial separation of cyclodextrin and acetylsalicylic acid or its derivative, that is to say a separation which can either be (a1) macrospatial, i.e. including the two compounds in two different phases separated from one other (preferably capsule shell and internal phase), or (a2) microspatial, i.e. dispersing both in the same oil phase contained within the capsule in amounts greater than their respective solubility limits, so as to have distinct particles of acetylsalicylic acid, or its derivatives, and distinct particles of cyclodextrin suspended in the same oil phase, and (b) the use of the complexing agent (cyclodextrin) in sub-stoichiometric quantities with respect to the active principle (acetylsalicylic acid or its derivatives).

It is surprising that despite the aforesaid “distance”, characterized by the macro- or microseparation established within the scope of the preferred embodiments of the invention described herein, cyclodextrins are nevertheless able to increase the stability of acetylsalicylic acid or its pharmaceutically acceptable derivatives against hydrolysis of the ester bond between the acetyl and salicylic groups.

Therefore, a first aspect of the present invention relates to the use of a compound of the cyclodextrin class for stabilizing formulations of acetylsalicylic acid or its pharmaceutically acceptable derivatives in soft capsules, comprising an internal liquid or semi-liquid oil phase containing acetylsalicylic acid partly dissolved and partly suspended therein, against the hydrolysis of acetylsalicylic acid or its pharmaceutically acceptable derivatives, characterized in that the compound of the cyclodextrin class is suspended in the same internal oil phase and/or is present in the shell of the soft capsule.

A second aspect of the present invention relates to a new formulation of acetylsalicylic acid or its pharmaceutically acceptable derivatives in soft capsules, in which acetylsalicylic acid or its derivative is partly dissolved and partly suspended in a liquid or semi-liquid oil phase contained within the soft capsule, a is compound of the cyclodextrin class being also suspended in the same liquid or semi-liquid oil phase.

A third aspect of the present invention relates to a new formulation of acetylsalicylic acid or its derivatives and omega-3 oils in a soft capsule, in which acetylsalicylic acid or its derivative is partly dissolved and partly suspended in a liquid or semi-liquid oil phase contained within the soft capsule, said liquid or semi-liquid oil phase comprising at least one omega-3 oil, characterized in that a compound of the cyclodextrin class is contained in the soft capsules shell, and that the content of EPA or DHA, or EPA and DHA together in the oil phase is at least 5% by weight, calculated as free acid.

### DETAILED DESCRIPTION OF THE INVENTION

As initially stated, the objective of the present invention is that of further increasing the stability of known formulations of acetylsalicylic acid or its derivatives in soft capsules. As seen above, said objective has now been attained by including cyclodextrins in formulations under conditions that do not favour (or that minimize) formation of inclusion complexes of cyclodextrins with acetylsalicylic acid or its derivatives within the soft capsule. Preferably the cyclodextrin quantities used are sub-stoichiometric relative to acetylsalicylic acid or its derivatives.

As is widely known from the literature, cyclodextrins (including for example hydroxypropyl-betacyclodextrin) are cyclic oligomers of glucose whose particular topology at the molecular level (truncated cone with a hydrophobic internal surface and a hydrophilic external surface) enables poorly hydrophilic compounds to be trapped inside them so that, after being captured within the truncated cone, they can be formulated in an aqueous environment due to the cyclodextrins having high water solubility. For example aqueous hormone formulations have long been proposed in which cyclodextrins act as excipients which solubilize the lipophilic hormone in aqueous environments.

The ability of cyclodextrins to incorporate hydrophobic compounds has been also exploited in another way. For example, U.S. Pat. No. 4,438,106 describes so-called inclusion compounds with EPA and/or DHA in the form of their alkaline salts or C1-C4 alkyl esters, being compounds which, as pharmaceutically acceptable oils, belong to the so-called omega-3 oil group preferred herein. Said inclusion compounds consist of a dry white odourless powder usable as a storage form for omega-3 acids protected against deterioration, or even, as such, as a raw material for the production of pharmaceutical formulations. U.S. Pat. No. 4,438,106 states that the complex must be formed in an aqueous-methanolic solution of the two components, heated to reflux and then cooled to 15° C. Decomplexing requires the solution of the complex in a water and hydrophilic solvent system, to be extracted with a non-polar organic solvent, followed by distillation of the non-polar solvent so as to recover the omega-3 acids or their derivatives.

Also described in the literature^{[4,5]} is an inclusion complex (1:1) of acetylsalicylic acid with beta-cyclodextrin. The authors hypothesize that in this case the acetylsalicylic acid would be positioned with its more hydrophobic part, i.e. the aromatic ring inside the cyclodextrin cavity, whilst exhibiting the carboxylic group and the acetylated phenolic group exteriorally.

An example of using this complex for obtaining a pharmaceutical formulation is described in CN 1460469 which is based on so-called oil-in-oil technology comprising firstly the preparation of an inclusion complex of acetylsalicylic acid in cyclodextrin followed by ultrasound-assisted dispersion of the complex within a first oil phase, then followed by emulsifying said first oil phase within a second phase. This approach, being rather laborious and aimed at obtaining pharmaceutical formulations that provide the consumer with a preformed inclusion complex, differs from the pharmaceutical forms herein described which instead hinder the formation of inclusion complexes of the utilized cyclodextrin with acetylsalicylic acid, as the applicant has found that cyclodextrins in the soft capsule formulations herein described are able to stabilize acetylsalicylic acid or its derivates from a distance. It has hence been possible to obtain formulations of acetylsalicylic acid or its derivatives of greater stability than known formulations.

To achieve the advantages of the present invention, it suffices to enclose the cyclodextrins either in the gelled material of the capsule's soft shell, or within the internal liquid oil phase comprising pharmaceutically acceptable oils, preferably including so-called omega-3 oils, in conditions which do not facilitate complexing of the acetylsalicylic acid or its derivatives; compounds of the cyclodextrin class can also be included either in the shell or in the internal oil phase. In all cases the stabilizing effect herein described is apparent even when cyclodextrins are used in very small, strictly sub-stoichiometric quantities relative to a hypothetical “1:1” inclusion complex of complexing agent with acetylsalicylic acid or its derivatives. Within this context, it should be noted that the use of specific cyclodextrins as a component of the gelatins used as pharmaceutical excipients, in particular for forming capsule shells, is known.

For example WO 99/33924 describes the use of beta-cyclodextrin—as an alternative to ethylenediaminetetraacetic acid, acetic acid, tartaric acid, metaphosphates and others—as an optional additive for improving the physical qualities of fish gelatins with previously added hydrocolloids (alginates, gum Arabic, starch, dextran and many others) which, without the addition of these latter, would be too mechanically fragile under conditions of use because of their glass-like characteristics and difficult to handle due to the liquid nature of the respective mixes at low temperatures.

WO 99/33924 hence uses cyclodextrins for other purposes and does not give any teachings on the possible effect that adding this additive, considered to be more or less optional, could have on the active principles contained in a liquid or semi-liquid oil phase within a soft capsule.

Japanese application no. 62 249935 also teaches to add specific cyclodextrins to the gelatinous shell of a soft capsule to modify certain characteristics thereof, in particular to maintain adequate disintegration in the stomach over time. Here also the objectives are different to those of the present invention; an effect on the stability of the active principles present in a distinct oil phase is neither described nor hypothesized.

Accordingly, the ability described herein of cyclodextrins to stabilize, from the shell, the acetylsalicylic acid present in an internal oil phase comprising pharmaceutically acceptable oils was not foreseeable in the light of prior knowledge.

Furthermore, as far as the applicant's knowledge extends, a suspension of cyclodextrins, as such, in the oil phase contained within a soft capsule was not known and neither was the possibility, in so doing, of stabilizing acetylsalicylic acid (or its derivatives) also present in the same oil phase in a quantity such that an undissolved excess remains.

From the above, the following aspects of the present invention are apparent, and are further illustrated below:
A first aspect of the present invention concerns the use of a compound of the cyclodextrin class for stabilizing formulations of acetylsalicylic acid or its pharmaceutically acceptable derivatives in soft capsules, in which the acetylsalicylic acid (or its pharmaceutically acceptable derivatives) is partly dissolved and partly suspended in a liquid or semi-liquid oil phase contained within the soft capsule, against the hydrolysis of acetylsalicylic acid or its pharmaceutically acceptable derivatives, characterized in that the compound of the cyclodextrin class is suspended within the same internal oil phase and/or is present in the soft capsule shell. Preferably the cyclodextrin suspended in the liquid or semi-liquid oil phase is present in a sub-stoichiometric quantity relative to the quantity of acetylsalicylic acid, or its pharmaceutically acceptable derivative, contained in the formulation. Preferably the cyclodextrin contained within the soft capsule shell is present in a sub-stoichiometric quantity relative to the quantity of acetylsalicylic acid, or its pharmaceutically acceptable derivative, contained in the formulation. More preferably, the total cyclodextrin is present in a sub-stoichiometric quantity relative to the quantity of acetylsalicylic acid, or its pharmaceutically acceptable derivative, contained in the formulation.
A second aspect of the present invention is the provision of a new formulation of acetylsalicylic acid or its pharmaceutically acceptable derivatives in a soft capsule, in which the acetylsalicylic acid or its derivative is partly dissolved and partly suspended in a liquid or semi-liquid oil phase contained within the soft capsule, and in the same liquid or semi-liquid oil phase there also being suspended a compound of the cyclodextrin class. Preferably the cyclodextrin suspended in the liquid or semi-liquid oil phase is present in a sub-stoichiometeric quantity relative to the quantity of acetylsalicylic acid, or its pharmaceutically acceptable derivative, contained in the formulation. Optionally, the shell of this new formulation can also contain a compound of the cyclodextrin class. In this case, the total cyclodextrin is preferably present in a sub-stoichiometric quantity relative to the quantity of acetylsalicylic acid, or its pharmaceutically acceptable derivative, contained in the formulation.
Finally, given that for the purposes of the present invention so-called omega-3 oils are preferably used as the pharmaceutically acceptable oil, a third aspect of the present invention relates to the provision of a new formulation of acetylsalicylic acid or its derivatives and omega-3 oils in a soft capsule in which the acetylsalicylic acid or its derivative is partly dissolved and partly suspended in a liquid or semi-liquid oil phase contained within the soft capsule, said liquid or semi-liquid oil phase comprising at least one omega-3 oil, characterized in that a compound of the cyclodextrin class is contained within the soft capsule shell and that the content of either EPA or DHA, or EPA and DHA together, in the internal liquid or semi-liquid oil phase, is at least 5% by weight calculated as free acid.

For the purposes of the third aspect of the present invention, it is also preferred that the cyclodextrin is present in a sub-stoichiometric quantity relative to the quantity of acetylsalicylic acid, or its pharmaceutically acceptable derivative, contained in the formulation.

For all three aspects of the invention, if the internal oil phase of the soft capsule contains omega-3 oils as the pharmaceutically acceptable oils, the content of EPA or DHA, or EPA and DHA together, in the liquid or semi-liquid oil phase within the soft capsule is preferably at least 5% by weight, more preferably at least 27% by weight and even more preferably at least 42% by weight calculated as free acid. In any event it is highly preferred that the content of EPA or DHA, or EPA and DHA together, is at least 50% by weight calculated as free acid. When EPA and DHA are together, they can coexist in any ratio though it is preferred that the EPA:DHA ratio is 1:2-2:1, preferably 0.9-2:1 and even more preferably 0.9-0.98:1. If EPA and DHA are present singly, the content of EPA or DHA, in the liquid or semi-liquid oil phase within the soft capsule is preferably at least 70% by weight, more preferably at least 80% by weight and even more preferably 90% by weight calculated as free acid.

The present invention as well as some of its preferred embodiments will now be explained in greater detail.

With regard to “acetylsalicylic acid or its derivates”, acetylsalicylic acid can either be used as such or as its pharmaceutically acceptable salts such as its lysine, ornithine, glycine or chitosan salt or for example its inorganic salts with Ca, Na, K, Al or others. For the purposes of the present invention, acetylsalicylic acid as such is preferred. Acetylsalicylic acid (or its derivatives) is used in the form of a powder or crystals of a particle size and quantity suitable for preparing a saturated suspension of the active principle in the internal liquid or semi-liquid oil phase of the capsule. Preferably powders are used where the fraction of particle size greater than 250 micron is less than 10%. Even more preferably the fraction of particle size greater than 250 micron is less than 1%. As an alternative, the acetylsalicylic acid can also be used in crystalline form, for example with a fraction of particle size greater than 125 micron of more than 60%, and a fraction of particle size greater than 355 micron of less than 5%.

With regard to the liquid or semi-liquid oil phase contained in the soft capsules of the present invention, into which the acetylsalicylic acid or its derivatives used in the present invention is introduced, the former contains at least one pharmaceutically acceptable oil as defined below. In addition to the pharmaceutically acceptable oil, the liquid or semi-liquid oil phase of the present invention can also comprise one or more optional excipients typically used to formulate the internal phase of soft capsules, such as thickeners (e.g. beeswax), emulsifiers (such as lecithin or glyceryl monostearate), surfactants (e.g. sorbitan derivatives such as polysorbate 20 or polysorbate 80), antioxidants (such as retinoic acid or derivatives, in particular retinyl palmitate, tocopherol or others) or diluents (such as linear or branched C2-C3 aliphatic alcohols or polyalcohols and C1-C2 esters thereof). Further optional usable excipients include colourings, opacifiers, flavourings, etc. The aforesaid optional excipients and other usable excipients with equivalent function are known to experts of this art.

With regard to “pharmaceutically acceptable oils”, this term comprises all vegetable, animal or synthetic oils commonly used in pharmaceutical formulations, as given in various manuals used by experts of the art, specifically the Pharmacopeia. For example, soya, sunflower or olive oils, or coconut or palm oils, or “mcts”, i.e. so-called medium chain triglycerides etc, either natural or modified e.g. hydrogenated, can be used. By way of example synthetic oils include silicone oils as permitted in the Pharmacopeia, such as dimethylpolysiloxanes (also-called simethicones or dimethicones).

In particular, the term “pharmaceutically acceptable oils” also includes oils, such as the so-called omega-3 oils, which can themselves perform the function of an additional active principle. For the purposes of the present invention the term “omega-3 oils” means natural or refined oils that comprise polyunsaturated fatty acids or their pharmaceutically acceptable derivatives, comprising from 18 to 22 carbon atoms, preferably from 20 to 22 carbon atoms, and in which the first double bond appears in the third position counting from the methyl end of the chain. In accordance with the specific convention adopted in the field of fatty acids, these are identified by the abbreviation X:Y:ω:z, in which X stands for the number of carbon atoms in the acid chain, Y stands for the number of double bonds, and z is the position number of the first carbon atom forming part of a double bond, starting from the methyl (or “w”) end of the chain. In the present invention, the structure of fatty acids in omega-3 oils is the following: X=18-22, Y=1-6, and Z=3, preferably X=20-22, Y=1-6 and Z=3. Therefore all pharmaceutically acceptable oils, comprising fatty acids or their pharmaceutically acceptable derivatives of formula C18-22:1-6:ω:3 are hence the omega-3 oils of the present invention. Vegetable oils such as linseed oil, but also oils of animal origin, such as fish oils, fall within this definition. Examples of preferred polyunsaturated fatty acids comprising from 20 to 22 carbon atoms of the present invention are EPA (C20:5 ω −3, i.e. eicosapentanoic acid) and DHA (C22:6 ω −3, i.e. docosahexanoic acid). In particular, all oils are preferred that comprise EPA, DHA or both, such as certain natural fish oils, their concentrates or their further purified forms (with the content of EPA+DHA being more than 75%, preferably more than 80% and even more preferably more than 90% by weight) as described for example in the Pharmacopeia, for use as active principles.

Although the fatty acids contained in the omega-3 oils can be fatty acids as such, or can be salified or esterified with C1-C4 monofunctional or polyfunctional alcohols i.e. for example methyl esters, ethyl esters or propyl esters, or as esters of ethylene glycol or glycerin, their use in the form of triglycerides is widely preferred, as they allow particularly stable preparations of the invention to be is obtained.

As already indicated, a characteristic of the present invention is that the internal liquid or semi-liquid oil phase of the capsule comprises the entire contents of the acetylsalicylic acid or its derivatives of the formulation. The acetylsalicylic acid or its derivatives is always added in a quantity in excess of the maximum solubility of acetylsalicylic acid (or its derivatives) in said phase, so that it is partly dissolved and partly suspended within it. The internal oil phase of the soft capsule can comprise 2-60% by weight of acetylsalicylic acid or its derivatives. Preferably the internal oil phase of the soft capsule comprises 5-40% by weight of acetylsalicylic acid or its derivatives, more preferably 7-35% by weight. Even more preferred is that the internal oil phase of the soft capsule contains 10%-32%, preferably 12%-27% by weight of acetylsalicylic acid or its derivatives.

Regarding the shell of the soft capsules described herein, this can be prepared in the traditional manner with components and excipients known to experts of this art. In particular, all the gelatins cited in the Pharmacopeia for this purpose can be used as the gelling component in the capsule shell, such as gelatin A or B (e.g. bloom 130-250), modified gelatin (e.g. succinylated) but also suitable substitute gelling compounds for example based on starch, carrageenan or certain polyphenyl compounds described in the literature (Eisai, Korean patent application 90-10411 of 10 Jul. 1990) or others. Particularly preferred is gelatin which can be of vegetable or animal origin, particularly of porcine, bovine, avian or fish origin. The gelling component is mixed with the additional excipients that are typically water and one or more non volatile plasticizers, which ensure the capsule's elasticity. These plasticizers are preferably polyhydroxy alcohols such as glycerin, propylene glycol, sorbitol, modified sorbitols, sorbitols/sorbitans, macrogol 200-600 or mixtures thereof. A commercial plasticizer is ANIDRISORB®, which is a mixture of sorbitol, sorbitans, maltitol and mannitol. Water serves as the solvent and provides the viscosity required for handling the gelatin mass, molten at a temperature of around 60-70° C. After capsule formation, the water content is reduced by drying. Additional optional excipients can be modifying excipients, such as partially hydrogenated starch hydrolysates, silicone oils (for example dimethicone) or other excipients used for formulating shells in the pharmaceutical field such as glyceryl behenate (COMPRITOL 8880), colourings, opacifiers, preservatives, antioxidants.

With regard to cyclodextrins, for the purposes of the present invention natural cyclodextrins such as alpha-, beta-, or gamma-cyclodextrins can be used; preferably modified cyclodextrins such as methyl-beta-cyclodextrin or sulfobutyl-beta-cyclodextrin, hydroxypropyl-gamma-cyclodextrin and hydroxypropyl-beta-cyclodextrin are used, in particular 2-hydroxypropyl-beta-cyclodextrin which is the most preferred herein.

With regard to the embodiments of the present invention which comprise cyclodextrins as a component of the gelatinous shell, it has been found that to preserve the processability and rheological characteristics required for the preparation of soft capsules in gelatin, the gelatinous mix initially used for capsule formation must not contain more than 20% by weight of cyclodextrin.

With regard instead to the embodiments of the present invention which comprise cyclodextrins as a component of the liquid or semi-liquid oil phase of the soft capsule, given their external hydrophilic characteristics cyclodextrins always form suspensions. Preferably the internal oil phase of the soft capsule comprises 1%-15% of cyclodextrins by weight, more preferably 2%-12% and even more preferably 3%-10%.

With regard to the production method for the new soft capsule formulations herein described, this is conventional and therefore can be undertaken with any machine commercially available for this purpose. An example of a well known procedure is the so-called rotary die process developed in 1932 by P. Scherer, with which forming, filling and sealing of soft capsules can be achieved in one operation. In said procedure, two ribbons derived from a molten gelatin mass (or other suitable gelling compound) are passed over two semi-dies of the desired shape, provided on the surfaces of two adjacent rotating rollers. As the die closes by means of rotation, the liquid content (i.e. the separately formed liquid or semi-liquid oil phase) is injected into the thus formed capsule by an injector, the capsule then being released as the die reopens.

The liquid or semi-liquid oil phase is obtained by known methods, by mixing the pharmaceutically acceptable oil with other components.

Additional procedures and more details are found in “Remington's Pharmaceutical Sciences”, 20th edition, edited by Alfonso R. Gennaro, 2000, Lipincott, Williams & Wilkins, ISBN 0-683-306472.

### Example 1

### Preparation of Soft Gelatin Capsules Containing ASA, Omega-3 and Cyclodextrin in Both the Fill and the Shell

### a) Preparation of the Mix for the Shell

Components and quantities for the preparation and relative percentages

| | | | |
|---|---|---|---|
| Gelatin 150 bloom | 26.4 | Kg | 33.0% |
| Propylene glycol | 1.6 | Kg | 2.0% |
| Anidrisorb 85/70 | 8.0 | Kg | 10.0% |
| Dimethicone 1000 | 7.2 | Kg | 9.0% |
| HPβ cyclodextrin | 12.0 | Kg | 15.0% |
| Purified water | 24.8 | Kg | 31.0% |

In a 150 litre turboemulsifier (Olsa-Italy) 24.8 litres of purified water are added to 12.00 kg of HPβ cyclodextrin (Kleptose HPβ from Roquette Freres—Fr) and maintained under agitation for 15-60 minutes at 30° C. When a clear solution is formed, 1.6 kg of propylene glycol, 7.2 kg of dimethicone and 8.0 kg of anidrisorb are added, again under agitation, and the temperature brought to 70° C.; 26.4 kg of gelatin are then added and maintained under agitation for 15-60 minutes. The mass is then deaerated by applying progressive vacuum until a value of between −0.8 and −0.9 bar is attained.

### b) Preparation of the Fill

Components and quantities for preparation and relative percentages

| | | |
|---|---|---|
| ASA | 1.920 Kg | 19.23% |
| Fish oil | 5.310 Kg | 53.07% |
| Yellow beeswax | 0.880 Kg | 8.85% |
| Hydrogenated coconut oil | 0.460 Kg | 4.615% |
| Refined palm oil | 0.460 Kg | 4.615% |
| HPβ cyclodextrin | 0.960 Kg | 9.615% |

In a 25 litre turboemulsifier (Olsa-Italy), 0.880 kg of beeswax, 0.460 kg of hydrogenated coconut oil, 0.460 kg of refined palm oil are mixed together; the temperature is brought to 70° C. and a vacuum of −0.85 bar applied. On attaining this temperature the fish oil is added, maintaining this temperature until the mass is completely molten. The mass is cooled to 25° C.±5° C., then the ASA and HPβ cyclodextrin are added followed by mixing for 30 minutes.

### c) Preparation of the Capsules

Soft gelatin capsules, size 8 oblong, were prepared in accordance with the process known as the Rotary Die Process.

Capsules with the following characteristics were obtained:
average weight per capsule: 654 mg±7.5%
residual moisture: 0.75%
ASA content: 72.825 mg/capsule equal to 97.1% d.d.
SA content: 0.2% on ASA
disintegration time, measured in accordance with the method codified by the European Pharmacopeia: <30 minutes.

### CITED LITERATURE

[1] “Hydrolysis of acetylsalicylic acid from aqueous suspensions”, K. C. James in J. Pharm. and Pharmacol. 10, 363-9 (1958).
[2] “Aspirin elixir” T. W. Schwarz et al. N. G. Shvemar et al. in J. Am. Pharm. Assoc., Pract. Pharm. Ed. 19, 40-1 (1958).
[3] “A review of the susceptibility of acetylsalicylic acid (ASA) to decomposition. Edward Stempel in Am. J. Pharm. 133, 226-34 (1961).
[4] Publication of the University of PV available from 11.07.2006 on the web “Cyclodextrins as agents for solubilizing drugs” http://chifar.unipv.it/dipchifar/betti/web/SolubilWWW2005_{—}06/CFA2005-06Solub9.htm
[5] “Dynamic study of interaction between beta-cyclodextrin and aspirin by the ultrasonic relaxation method.” Fukahori et al. in J. Phys.Chem.B. Condens Matter Surf Interfaces Biophys. 2006, Mar. 9, 110(9):4487-91.

1-55. (canceled)

56. Pharmaceutical formulation of acetylsalicylic acid or its pharmaceutically acceptable derivatives in a soft capsule made of an outer shell and an inner liquid or semi-liquid oil phase wherein the acetylsalicylic acid or its derivative is partly dissolved and partly suspended in the said liquid or semi-liquid oil phase, a compound of the cyclodextrin class being suspended in the said liquid or semi-liquid oil phase, and/or contained in said shell, in order to provide stability to the formulation.

57. Formulation as claimed in claim 1 wherein the cyclodextrin is suspended in the liquid or semi-liquid oil phase in a sub-stoichiometric quantity with respect to the quantity of acetylsalicylic acid or its pharmaceutically acceptable derivative.

58. Formulation as claimed in claim 1 wherein the cyclodextrin is contained in the soft capsule shell in a sub-stoichiometric quantity with respect to the quantity of acetylsalicylic acid or its pharmaceutically acceptable derivative.

59. Formulation as claimed in claim 1 wherein the total cyclodextrin is present in a sub-stoichiometric quantity with respect to the quantity of acetylsalicylic acid or its pharmaceutically acceptable derivative.

60. Formulation as claimed in claim 1 wherein the said oil phase comprises 2-60% by weight of acetylsalicylic acid or its derivatives, preferably 5-40% by weight and more preferably 7-35% by weight.

61. Formulation as claimed in claim 1 wherein the said oil phase comprises 1-15% by weight of a compound of the cyclodextrin class, preferably 2-12% by weight and more preferably 3-10% by weight.

62. Formulation as claimed in claim 1 wherein the said shell comprises gelatin and/or modified gelatin and/or a suitable substitute gelling compound, water and/or a plasticizer and optionally one or more excipients.

63. Formulation as claimed in claim 1 wherein the said liquid or semi-liquid oil phase contains at least one omega-3 oil.

64. Formulation as claimed in claim 8 wherein the omega-3 oil comprises fatty acids or their pharmaceutically acceptable derivatives of formula C18-22:1-6:ω:3 including EPA and/or DHA.

65. Formulation as claimed in claim 9 wherein the content of EPA or DHA, or EPA and DHA together, is at least 5% by weight, calculated as free acids.
11. Formulation as claimed in claim 10 wherein the content of EPA or DHA, or EPA and DHA together, is at least 27% by weight, more preferably at least 42% by weight and even more preferably at least 50% by weight, calculated as free acids.

66. Formulation as claimed in claim 9 wherein the EPA:DHA ratio is 1:2-2:1, preferably 0.9-2:1 and even more preferably 0.9-0.98:1.

67. Formulation as claimed in claim 1 wherein the said compound of the cyclodextrin class is chosen in the group consisting of natural cyclodextrins such as alpha-, beta- or gamma-cyclodextrins, and modified cyclodextrins such as methyl-beta-cyclodextrin, sulfobutyl-beta-cyclodextrin, hydroxypropyl-gamma-cyclodextrin and hydroxypropyl-beta-cyclodextrin.

68. Formulation as claimed in claim 9 wherein the said liquid or semi-liquid oil phase comprises EPA and/or DHA, 2-hydroxypropyl-beta-cyclodextrin in suspension, and acetylsalicylic acid partly dissolved and partly suspended in the said liquid or semi-liquid oil phase.

69. Formulation as claimed in claim 1, wherein the acetylsalicylic acid or its derivative is partly dissolved and partly suspended in the said oil phase, wherein a compound of the cyclodextrin class is contained in the soft capsule shell and in the said oil phase the content of EPA or DHA, or EPA and DHA together, is at least 5% by weight, calculated as free acids.
